(19) United States
(12) Patent Application Publication
(10) Pub. No.: US 2002/0090698 A1
(43) Pub. Date: Jul. 11, 2002

(54) RECOMBINANT PROCESS FOR THE PRODUCTION IN PSEUDOMONAS PUTIDA OF THE CYTOCHROME C551 OF PSEUDOMONAS AERUGINOSA

(76) Inventors: ALFREDO COLOSIMO, ROMA (IT); FRANCESCA CUTRUZZOLA', ROMA (IT); ILARIA MARIA CIABATTI, ROMA (IT); ELISABETTA ZENNARO, ROMA (IT); CARLO VISCO, MILANO (IT); MASSIMO DISCEPOLO, MILANO (IT)

Correspondence Address: NIKAIDO MARMELSTEIN MURRAY & ORAM, METROPOLITAN SQUARE, 655 FIFTEENTH STREET N W, SUITE 330 G STREET LOBBY, WASHINGTON, DC, 200055701

(21) Appl. No.: 09/101,807

(22) PCT Filed: Mar. 10, 1997
(86) PCT No.: PCT/EP97/01213

(30) Foreign Application Priority Data
Mar. 15, 1996 (IT) MI96A000515

(51) Int. Cl. C12N 9/02; C07H 21/04; C12P 21/06; C12N 1/20; C12N 15/00; C12N 15/09; C12N 15/63; C12N 15/70; C12N 15/74

(52) U.S. Cl. 435/189; 435/69.1; 536/23.5; 435/320.1; 435/252.3; 435/252.34

(57) ABSTRACT

The invention relates to a recombinant process for the production of a hemoprotein having the ability of transporting electrons and comprising the sequence of cytochrome C₅₅₁ of Pseudomonas aeruginosa, characterized in that this hemoprotein is produced in Pseudomonas putida. The hemoprotein obtained through the process of the invention includes the natural form of cytochrome C_{551,} as well as its precursors characterized by the presence of all or a portion, typically an N-terminal part, of a signal sequence. The hemoprotein object of the invention can be prepared in the following way: (a) providing a host, transformed with the expression vector comprising a DNA sequence which encodes the hemoprotein, in such conditions that said hemoprotein is expressed and (b) isolating or purifying said hemoprotein. Hence further objects of this invention also includes: an expression vector comprising a DNA sequence which encodes a hemoprotein of the invention; a host transformed with a suitable expression vector according to the invention; and a DNA molecule of natural or synthetic origin, comprising a sequence encoding the hemoprotein according to the present invention. A hemoprotein according to the invention finds useful applications in the diagnostic field, for example as chromogenic substrate for perioxidase or in electrochemical studies, in which it is used for the detection, measurement and control of the reactions of electronic transfer between oxidoreductive proteins and electrode.

The present invention relates to a recombinant process for the production of cytochrome C₅₅₁ of Pseudomonas aeruginosa in the bacterial system of Pseudomonas putida.

Cytochrome C₅₅₁ is an electron-transport hemoprotein extracted from the bacterium Pseudomonas (Ps) aeruginosa (Horio et al., 1960).

Most likely its physiologic role consists of supplying electrons to nitrite reductase, a key enzyme of dissimilative denitrification which reduces nitrite to NO, but it is also able to very rapidly exchange electrons with azurin, an electron-transport protein containing copper which participates in the same and perhaps other metabolic pathways in Ps. aeruginosa.

Cytochrome C₅₅₁ is well characterized from the structural (tridimensional structure) and functional (transfer of electrons with small redox molecules and physiologic macromolecular partners) standpoint and this makes it a macromolecule most suitable for in-depth studies regarding the role that the protein matrix plays in controlling the reactivity of the prosthetic heme group and velocity and direction of the processes of electron transfer, possibly also through site-specific mutagenic studies.

For this purpose, it would be most useful to have an efficient system of protein expression available, which makes it possible to easily obtain relevant quantities of native cytochrome C₅₅₁ and which can then be used also to express site-directed mutants.

The expression of the cytochromes of type c poses several problems: i) the heme group is linked to the protein by two covalent bonds whose formation is catalyzed by a specific enzyme; ii) the cytochromes c incorporate heme after having reached a specific cellular compartment (intermembrane space of the mitochondrium for eukaryotic cytochromes, periplasmic space for the prokaryotes), towards which they are translocated by a characteristic signal sequence present in the protein, subsequently removed by a specific protease.

To date expression of this class of proteins has been undertaken in the following cases:

a) mutant yeast cytochromes expressed in the yeast itself by the substitution of the encoding gene in the chromosome (Margoliash et al., 1990); this approach allows to express only mutants of yeast cytochrome which maintains the function of the protein itself at an acceptable level, since the growth of the cell is linked to the presence and activity of this cytochrome;

b) expressions of cytochrome C₅₅₀ of Th. versutus in E. coli (Ubbink et al., 1992); this approach has enabled expression of a bacterial cytochrome with reasonable yields in a heterologous system, but it requires an almost anaerobic environment in order that E. coli produce a sufficient quantity of heme. This condition is experimentally not defined quantitatively and less simple to obtain compared to aerobic culture conditions, especially on a medium-large scale.

These problems are solved by the present invention which relates to the expression of cytochrome C₅₅₁ of Ps. aeruginosa produced in Ps. putida. This bacterial species has been selected since it grows naturally in aerobic conditions and presents limited nutritional demands, thereby enabling possible growths in the fermenter with restricted costs; in addition, it normally expresses type c cytochromes and this ensures the presence and efficiency of the systems of heme incorporation. The approach selected proved to be effective, with elevated expression levels of cytochrome C₅₅₁ with properties indistinguishable from that of the native one and absence of cell toxicity, and which might be applied also to other bacterial cytochromes c and possibly to eukaryotes. The physico-chemical characteristics of cytochrome C₅₅₁ have in addition made it possible to develop a procedure for the purification of the recombinant protein, particularly easy, rapid and economic.

Thus, it constitutes a first object of the present invention a recombinant process for the production of a hemoprotein having the ability of transporting electrons and comprising the sequence of cytochrome C₅₅₁ of Pseudomonas aeruginosa, characterized in that this hemoprotein is produced in Pseudomonas putida. As mentioned, cytochrome C₅₅₁ is a hemoprotein which acts as an electron transport system; it thus finds useful applications in the diagnostic field, for example as chromogenic substrate for peroxidase or in electrochemical studies, in which it is used for the detection, measurement and control of electronic transfer reactions between oxidoreductive proteins and the electrode.

The hemoprotein obtained by the process of the invention includes the natural form of cytochrome C₅₁₁, as well as its precursors characterized by all or a portion, typically an N-terminal part, of a signal sequence which has the function of directing the cytochrome towards the cellular compartment where incorporation of heme takes place and where the protein exerts its functional activities. This signal sequence can be both that of the native natural protein or it can be an exogenous signal sequence.

The hemoprotein object of the invention can be prepared in the following manner:

a) providing a host, transformed with an expression vector comprising a DNA sequence which encodes this hemoprotein, in such conditions that said hemoprotein is expressed; and

b) isolating or purifying said hemoprotein.

This represents a second aspect of the present invention. This approach is typically based on the construction of a nucleotide sequence which encodes the hemoprotein that is desired to be expressed and on the expression of the hemoprotein in a recombinant host organism. The culture of the genetically modified organism leads to the production of the desired protein endowed with biologic activity. Thus, further objects of the present invention consist also of,

an expression vector comprising a DNA sequence which encodes a hemoprotein of the invention,

a host transformed with a suitable expression vector according to the invention; and

a DNA molecule of natural or synthetic origin, comprising a sequence encoding the hemoprotein according to the present invention.

A host in which a hemoprotein can be expressed according to the invention is prepared by transforming a host with a compatible expression vector according to the invention. The expression vector can be prepared by:

a) enzymatically synthesizing a DNA sequence which encodes the hemoprotein of the invention starting from a part of the genome of Ps. Aeruginosa (Silvestrini et al., 1989); and

b) inserting said DNA within an expression vector.

Alternatively, an expression vector can be prepared by:

a) isolating from the Ps. aeruginosa genome the gene coding for the cytochrome C₅₅₁; and

b) inserting said gene in the expression vector.

Accordingly, the hemoprotein according to the present invention is prepared providing a transformed host and cultivating this host in such conditions in which the hemoprotein can be expressed.

In order to produce a hemoprotein of the invention by means of the recombinant DNA technique, a gene which encodes said hemoprotein is prepared. The invention includes a DNA molecule consisting essentially of the following sequence

| | |
|---|---|
| GAA GAC CCC GAA GTG CTG TTC AAG AAC AAG GGC TGC GTG GCC | [SEQ ID NO:1] |
| TGC CAT GCC ATC GAC ACC AAG ATG GTC GGC CCG GCC TAC AAG GAC | |
| GTC GCC GCC AAG TTC GCC GGC CAG GCC GGC GCG GAA GCG GAA | |
| CTC GCG CAG CGG ATC AAG AAC GGC AGC CAG GGC GTC TGG GGC | |
| CCG ATC CCG ATG CCG CCG AAC GCG GTC AGC GAC GAC GAG GCG | |
| CAG ACC CTG GCG AAG TGG GTC CTG TCG CAG AAA TGA | |

The coding DNA sequence typically does not contain introns.

The gene encoding cytochrome C₅₅₁ can be isolated from the operon in which it is naturally present by means of the PCR technique (Polymerase Chain Reaction, Mullis and Faloona, 1987).

For this purpose oligonucleotides complementary to the 5′ terminal end and 3′ terminal end of the gene coding for cytochrome C₅₅₁, can be synthesized and then used for cloning in the expression vector exploiting the restriction sites present therein. Typically, the expression vector includes appropriate transcription and translation control elements, such as a promoter for the gene to be expressed, a transcription terminal site as well as translation start and termination codons. The gene is presented in the correct structure so as to enable expression of the hemoprotein in a host compatible with the vector. The expression vector typically comprises an origin of replication and possibly a marker gene such as a gene conferring resistance to an antibiotic.

As mentioned, the expression vector is used to transform a suitable host which is cultivated in such a way as to ensure that the expression occurs.

The transformed host can be either a prokaryote or an eukaryote. In particular, bacterial hosts can be used. A preferred batcterial host is Ps. putida.

The hemoprotein that is expressed can be isolated and purified. As mentioned above, the hemoprotein can include a transport signal sequence. The transport sequence is typically present at the N-terminal end of the hemoprotein of the invention.

A hemoprotein according to the invention can be typically used for diagnostic applications. In particular, an area of intense research activity is represented by studies on metalloproteins electrons transfer reactions. A particular approach to these studies consisted of the use of electrochemical methods to investigate the electron transfer reactions which take place at the interface between the electrode and the solution. Thus, electrodes able to react with various types of cytochromes have been devised: in particular, the electrochemistry of cytochrome C₅₅₁ of Ps. aeuginosa has been studied through the use of gold electrodes modified with polyfunctional organic molecules and it has thus been demonstrated that cytochrome C₅₅₁ is able to exchange electrons with this electrode (H. Allen O. Hill, et al., J. Elletroanal. Chem., 217 (1987): 129-140).

This information has suggested the use of cytochrome C₅₅₁ in the construction of biosensors able to detect enzymatic reactions which directly involve this molecule. These biosensors prove useful in assay methods for the determination of enzymes and substrates or in clinical procedures. For example, they have been used for the determination of glucose present in biological fluids, in particular in diabetics (European patent application No. EP125137) or for the detection of H₂O₂ in active or passive systems, in particular to control the activity of redox enzymes such as glucose-oxidase, oxalate-oxidase and cholesterol-oxidase (British patent No. GB 2206414).

The following examples illustrate the invention. In the attached drawings:

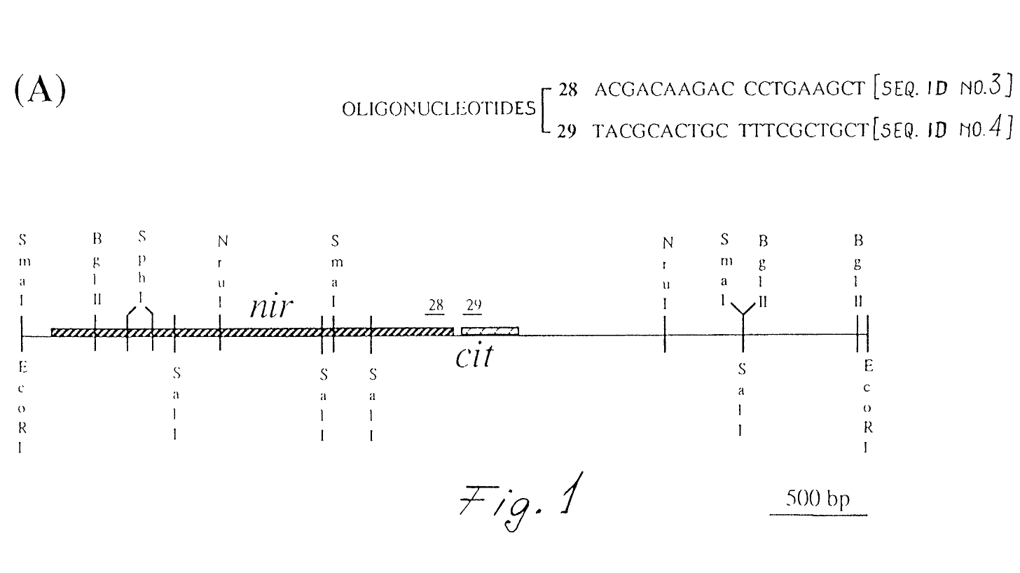

FIG. 1. (A) Sequence of two oligonucleotides [SEQ ID NOs. 3 and 4] used to verify the presence of the cit gene in the 3.5 kb genomic DNA fragment of Ps. aeruginosa, previously characterized (Silvestrini et al., 1989). Restriction map of the fragment and localization on the fragment itself of the oligonucleotide sequences and of the nir and cit genes.

(B) DNA sequence obtained by the Sanger method using as primers the oligonucleotides 28 [SEQ ID NO. 3] and 29 [SEQ ID NO. 4]. The zones encoding the end of the nir gene and the beginning of the cit gene are translated into aminoacids.

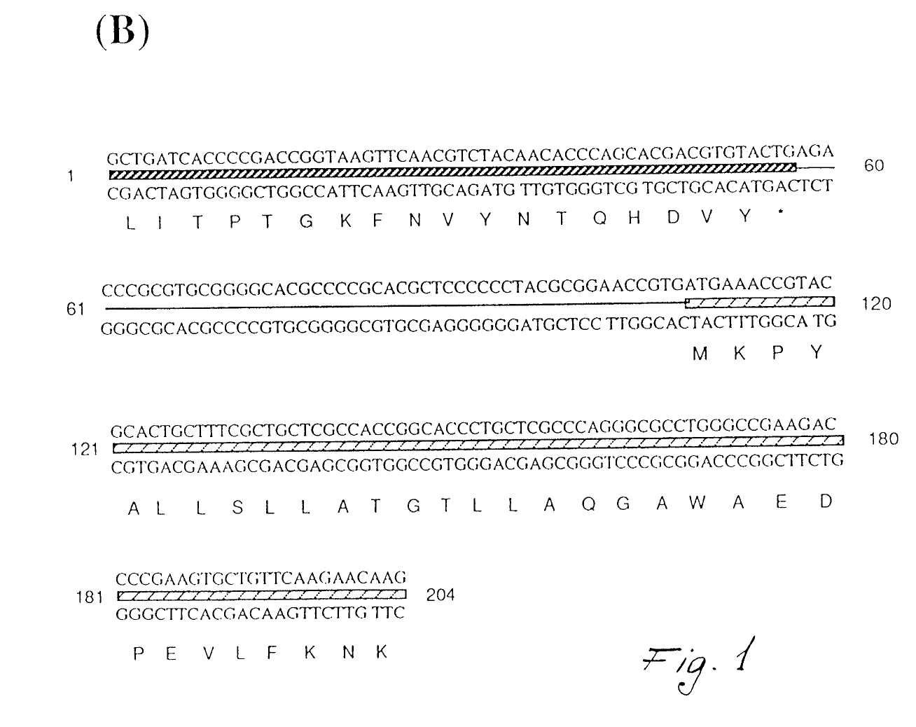

FIG. 2. Oligonucleotides used for the selective amplification through PCR of cit gene starting from the fragment of 3.5 kb described in FIG. 1A and for the subsequent cloning in vector pNM185 (oligo NM-1 [SEQ ID NO. 5] and NM-2 [SEQ ID NO. 6]).

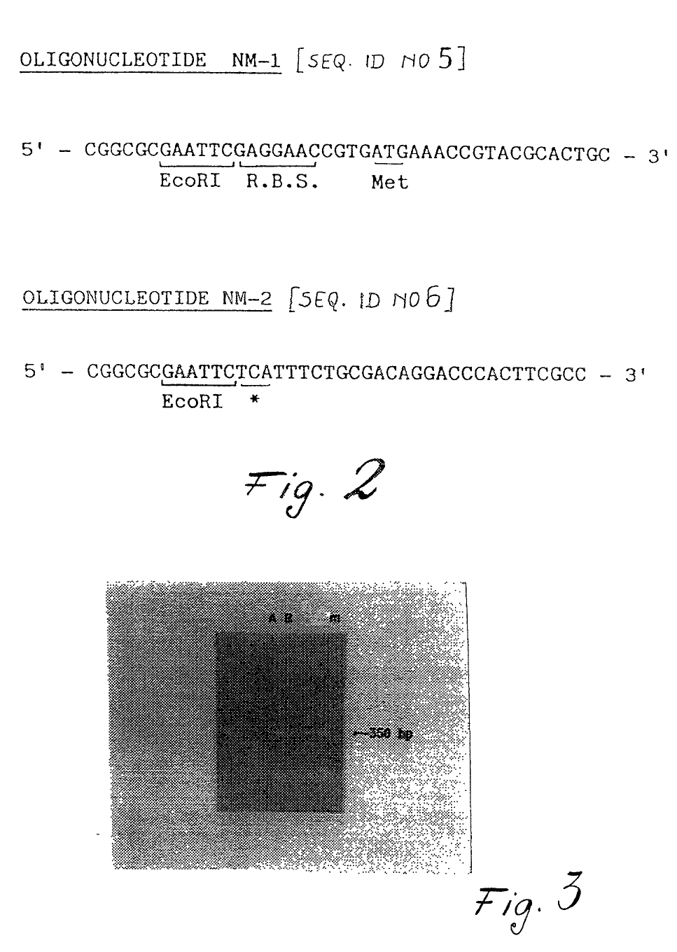

FIG. 3. Agarose gel (1.2%) analysis to verify the amplification through PCR of the cit gene.

A and B: 1 and 10 ng of pEMBL18NR amplified with primers NM-1 and NM-2.

m: lambda phage digested with Hindlil as molecular weight marker.

The arrow indicates the fragments amplified and their size.

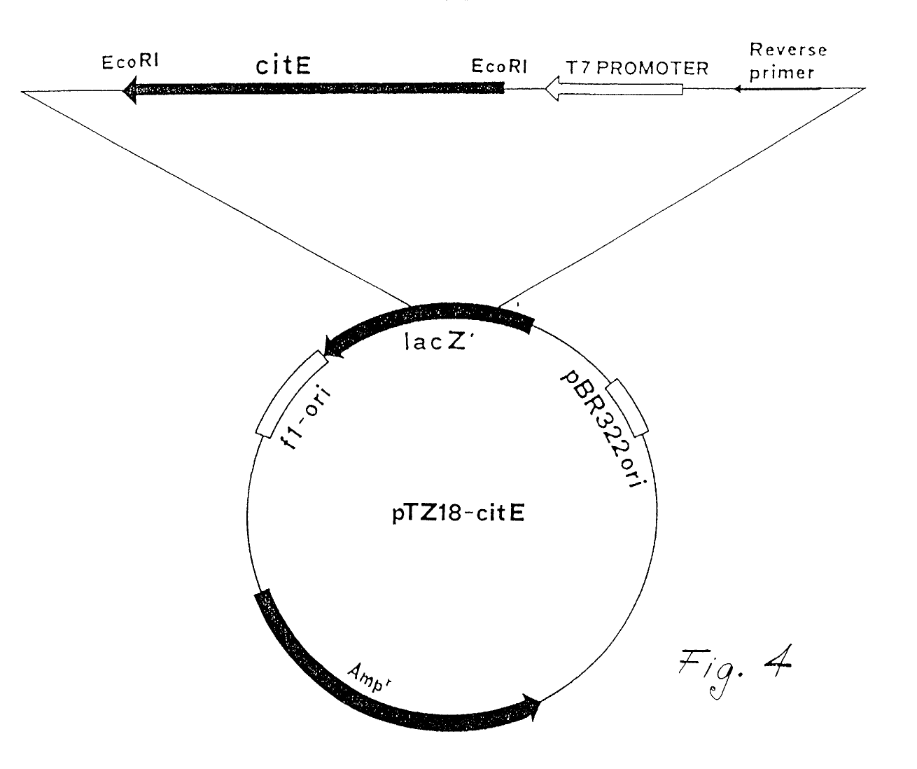

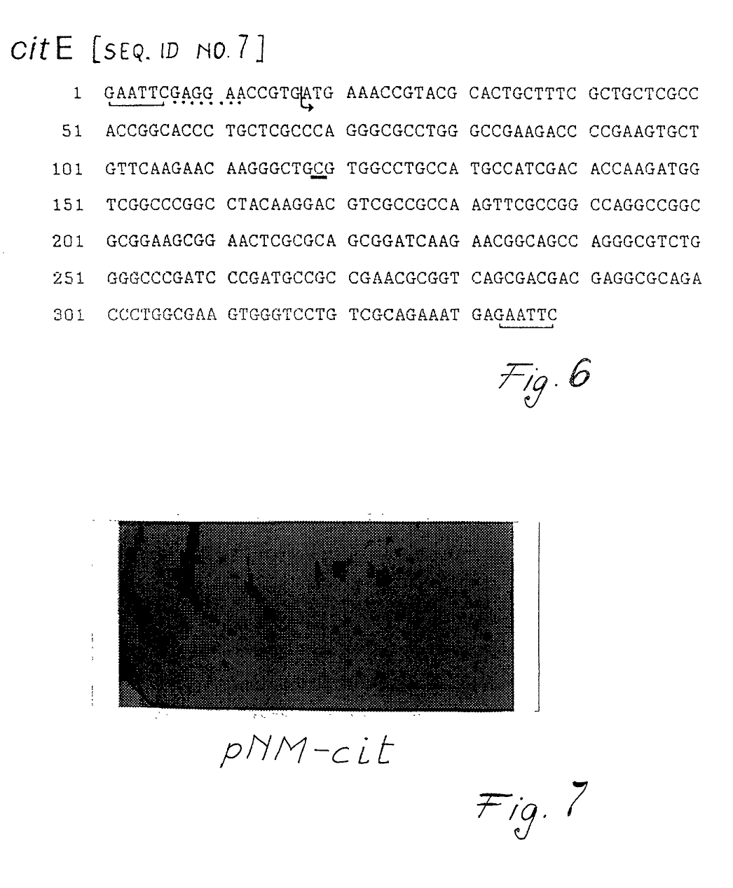

FIG. 4. Map of the clones used to determine the sequence amplified by PCR. The sequence of the insert citE is shown in FIG. 6.

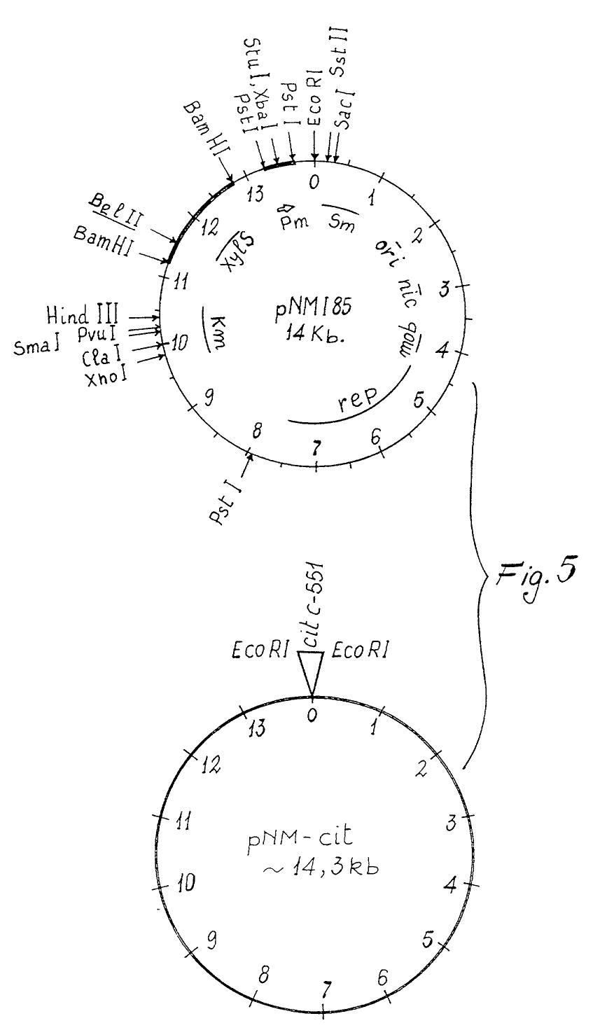

FIG. 5. Map of the vector pNM185 and of the clone derived therefrom used for the expression in Ps. putida amplified by PCR

FIG. 6. Sequence of the insert citE [SEQ ID NO. 7] containing the gene of cit C₅₅₁. The ribosome binding site RBS ( . . . ), the starting codon (ATG) and the only silent substitution detected in position 102 of the coding sequence are indicated.

FIG. 7. Autoradiography of colony hybridization for the isolation of the recombinant plasmid pNM-cit.

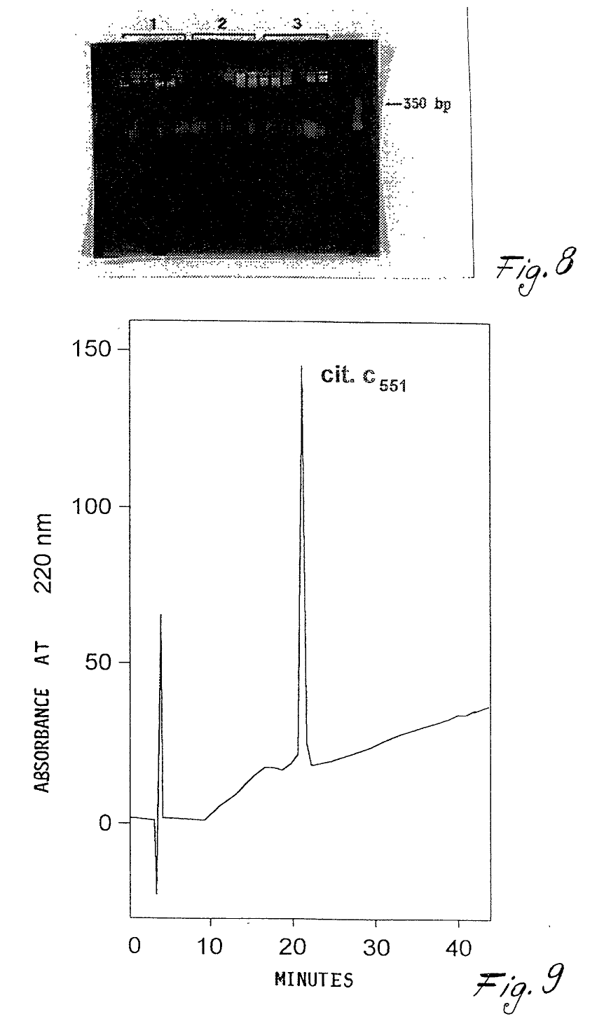

FIG. 8. Agarose gel (1.2%) analysis to verify the isolation of recombinant plasmid pTZ 18-citE (1).

FIG. 9. Reversed-phase high pressure liquid chromatography analysis of a purified preparation of recombinant C₅₅₁ cytochrome.

The separation was carried out on a C18 column with linear gradient of acetonitrile in water- trifluoroacetic acid and with detection at 220 nm wavelength.

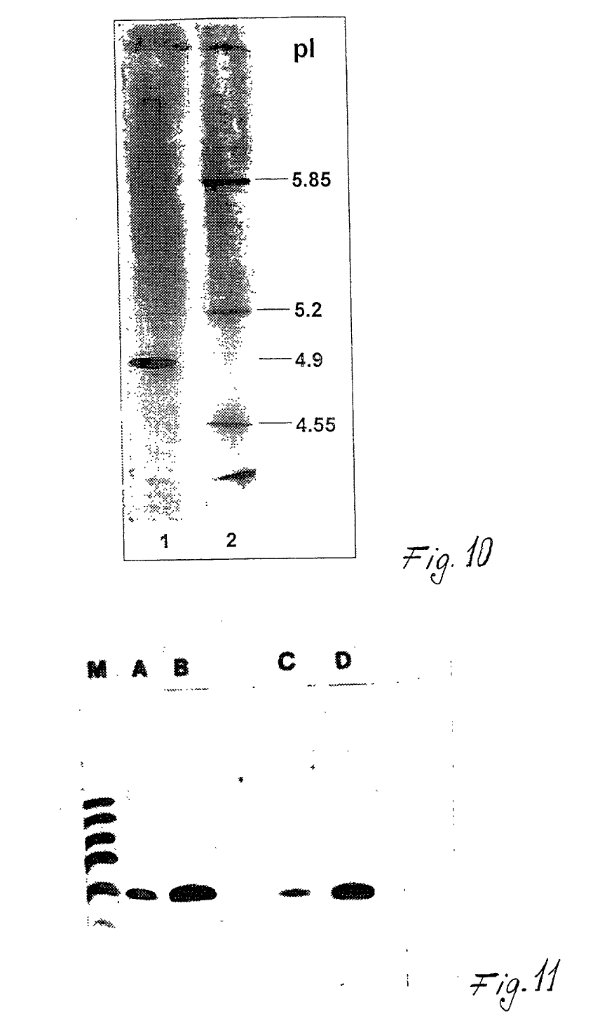

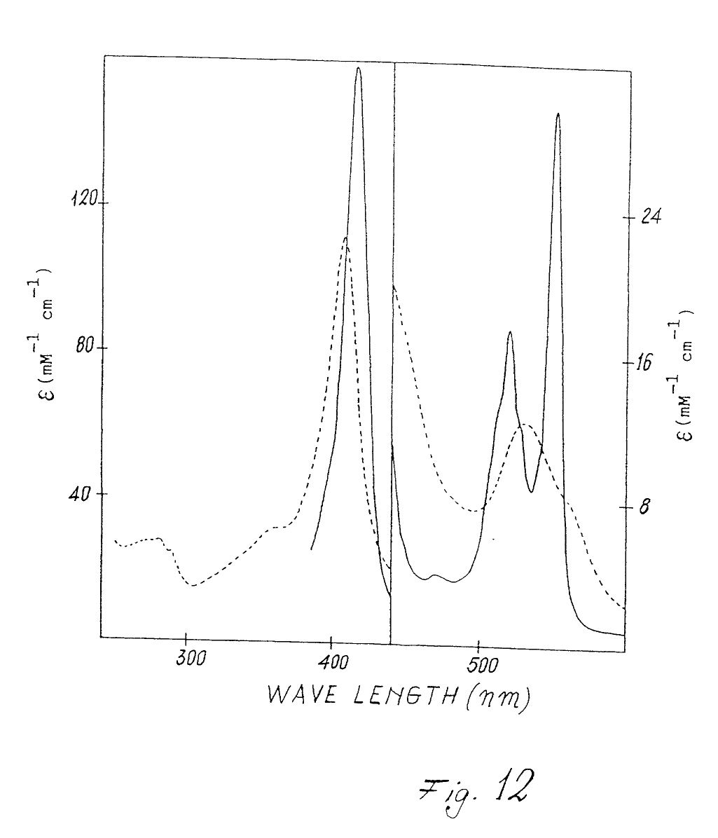

FIG. 10. Analysis of isoelectric focusing of a purifed preparation of cytochrome C₅₅₁.

The separation was carried out on polyacrylamide gel (Immobiline dry Plate) with pH gradient from 4 to 7. The recombinant cytochrome C₅₅₁ focuses in a homogeneous protein band with pI=4.9 (sample 1). The isoelectric point was estimated by comparison with a mixture of standard Biorad proteins (sample 2).

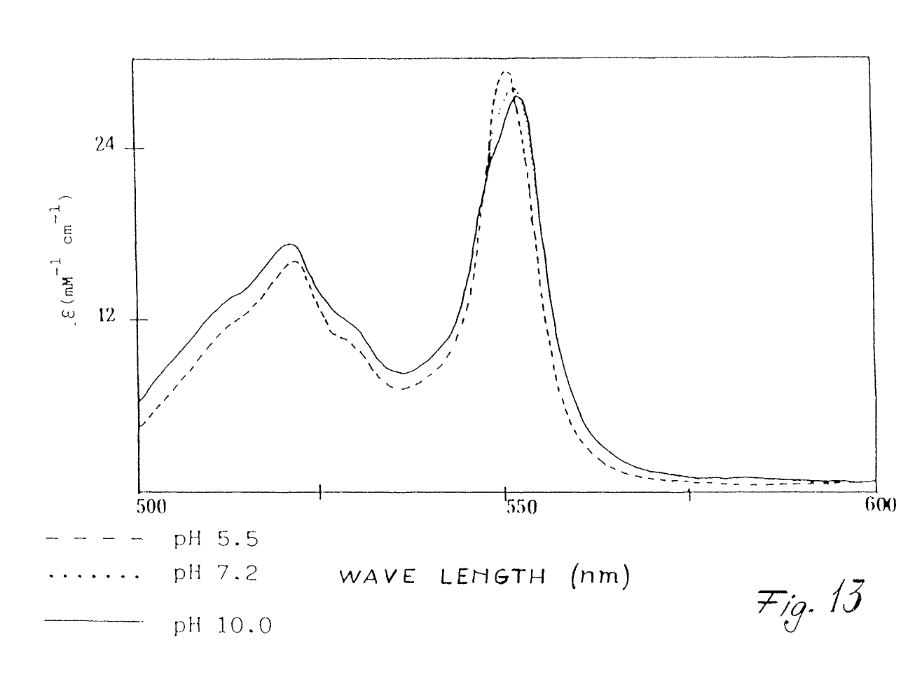

FIG. 11. Polyacrylamide gel in SDS-tricine. A and B: increasing amounts of purified cytochrome C₅₅₁ from Ps. aeruginosa; C and D: increasing amounts of purified cytochrome C₅₅₁ from Ps. putida; M: molecular weight markers (range 10-100 kDa).

FIG. 12: Spectra of purified cytochrome C₅₅₁ from Ps. putida, in the oxidized form (dashed line) and reduced form (continuous line).

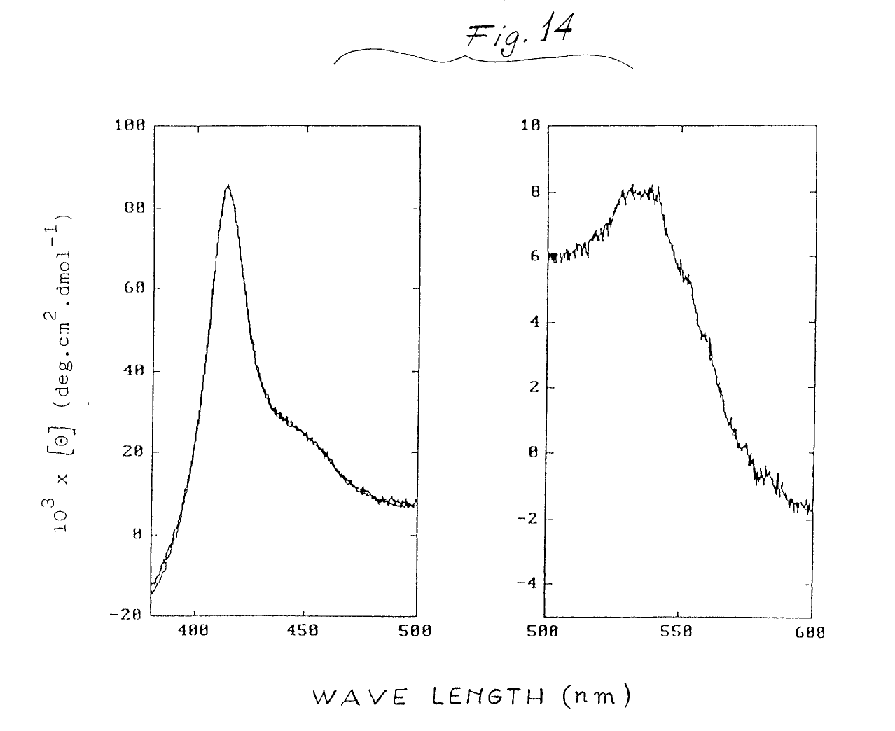

FIG. 13. Spectra in the visible of purified cytochrome C₅₅₁ from Ps. putida: the protein in the reduced form was analyzed at the three reported pH values.

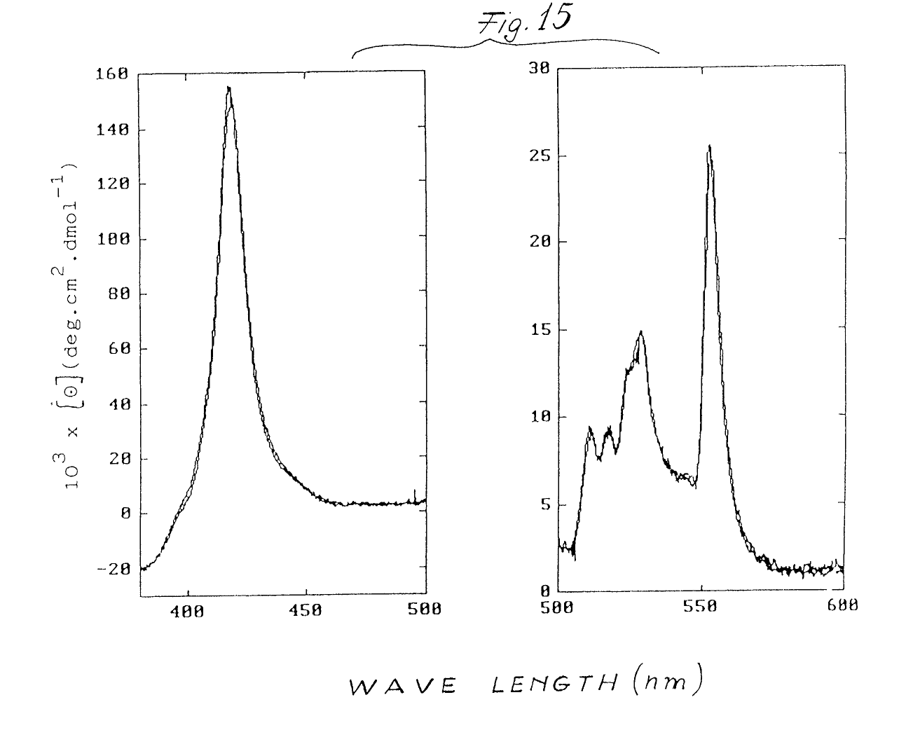

FIG. 14. Spectra of circular dichroism of purified cytochrome C₅₅₁ from Ps. putida: the spectra of a solution of oxidized cytochrome are shown.

FIG. 15. Spectra of circular dichroism of purified cytochrome C₅₅₁ from Ps. putida: the spectra of a solution of reduced cytochrome are shown.

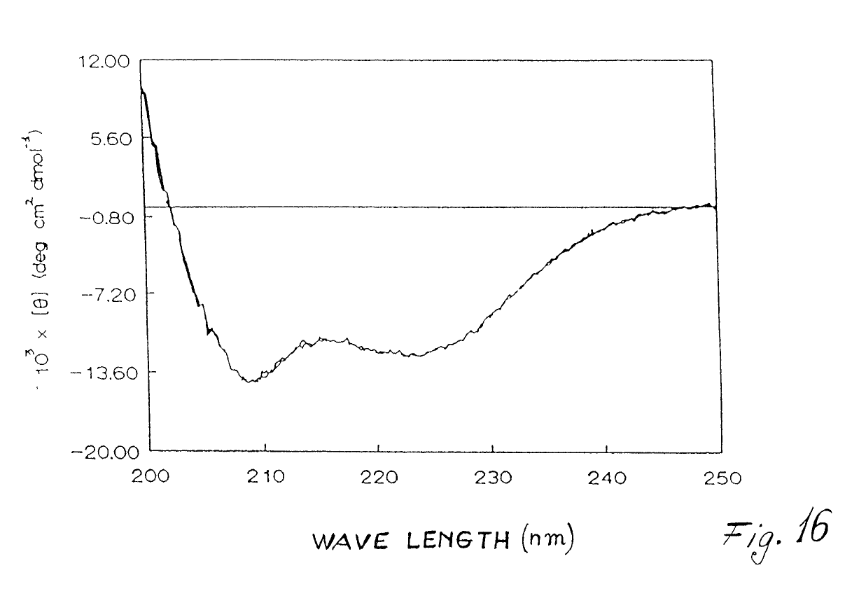

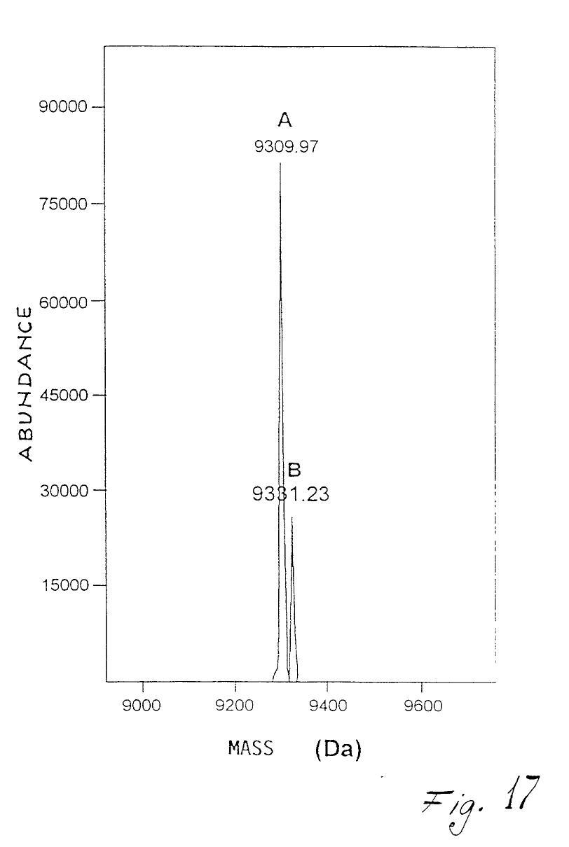

FIG. 16. Spectra of circular dichroism in the deep UV region. The spectrum of a solution of oxidized cytochrome is shown.

FIG. 17. Mass spectrometry analysis of a purified preparation of recombinant cytochrome C₅₅₁.

Peak A with mass of 9309.97 Da corresponds to the mass calculated of cytochrome C₅₅₁; peak B corresponds to an adduct cytochrome C₅₅₁-sodium ion formed in the ionization conditions

### EXAMPLE 1

### Identification of the Cit Gene in the Operon

Recent literature data (Nordling et al.; 1990; Arai et al.; 1990) have reported the isolation of the gene coding for cytochrome C₅₅₁ (cit) in an operon of the Ps. aeruginosa bacterium genome; starting from this information, the presence of this gene was checked in a fragment of genomic DNA 3.5 kb long, previously isolated (Silvestrini et al., 1989) and containing the gene coding for the nitrite reductase enzyme (nir).

For this purpose specific oligonucleotides were synthesized (designated primers 28 [SEQ ID NO. 3]and 29 [SEQ ID NO. 4]) complementary to the 3′ end of the nir gene and to the 5′ end of the cit gene, respectively, and the nucleotide sequence of a segment of 200 bases was determined: this sequence, compared with that reported in the literature, revealed the presence of the cit gene in the genomic DNA fragments under study.

The sequence of the primers used, their localization inside the previously characterized fragment and the sequence of the segment of genomic DNA are reported in FIG. 1.

### Methods Employed

Except for the case explicitly indicated with specific references, all the methods of molecular genetics employed herein are described in Sambrook et al. (1989).

1) Preparation of DNA for the determination of the sequence.

A single-stranded DNA (ssDNA) is prepared by infecting a culture of DH5 bacterial cells with the DNA of plasmid pEMBL18-NR (Silvestrini et al., 1989), in the presence of bacteriophage F1.

The infected culture produces ssDNA in the medium: this is recovered by precipitation with PEG/NaCl and controlled by electrophoresis on 1% agarose gel in TBE medium.

2) Construction of Primers 28 and 29.

The nucleotide sequence of primer 28 [SEQ ID NO. 3] was decided on the basis of previous information (Silvestrini et al., 1989) whereas that of primer 29 [SEQ ID NO. 4] was designed after determination of the first segment of the new sequence. The sequences are reported in detail in FIG. 1.

3) Determination of the nucleotide sequence.

The sequence was determined by the method of Sanger et al. (1977) using Sequenase version 2.0 reagents (USB) both with dGTP and dITP to eliminate problems of compression deriving from the high content in GC of the DNA of Ps. aeruginosa. The sequence reactions were resolved by means of electrophoresis on 6% urea/polyacrylamide gel.

### EXAMPLE 2

### Isolation and Cloning of the Cit Gene

Once the presence of the cit gene was identified in the operon, isolation of the gene from the remaining part of the operon was undertaken. This operation was deemed necessary for a series of reasons:

the presence of the flanking sequences (including that coding for nitrite reductase) complicates the nutritional requirements of the transformed Ps. putida strain, since the culture medium must be enriched with compounds such as KNO₃ and lowers the yield in biomass as a result of a toxic effect of overexpression of the nitrite reductase;

the efficiency of transcription starting from promoter Pm of expression vector pNM185 is reduced due to the position of the cit gene located at the 3′ end of the nir gene, which is instead found immediately downstream of the promoter;

the protocol for purification is also complicated as a result of co-expression of nitrite reductase.

In order to obtain better yields of expression and to simplify the protocol of bacterial growth and purification of the recombinant protein, the cit gene was isolated from the context of the operon using PCR (Polymerase Chain reaction, Mullis and Faloona, 1987).

Synthetic oligonucleotides (FIG. 2) were designed and synthetized, suitable for subsequent cloning of the cit gene in vector pTZ18 (for the determination of the nucleotidic sequence) (Mead et al., 1986) and pNM185 (for the expression in Ps. putida) (Mermod et al., 1986).

In the case of vector pNM185, which possesses only the transcription starting signals, in addition to the sequence recognized by EcoRI, also the RBS (Ribosome Binding Site) was inserted in the synthetic oligonucleotides of the cit gene needed for initiation of translation.

For the cloning in the sequence vector, the same oligonucleotides described for cloning in pNM185 were used.

The result of the PCR reaction is a fragment of approximately 350 nucleotides, subsequently purified and inserted in the above described vectors.

The recombinant plasmids were inserted by transformation in E,coli JM109 and isolated in single colonies by hybridization with a radioactive probe corresponding to the cit gene.

The restriction maps of the different constructs are reported in FIGS. 4 and 5: the recombinant plasmids were designated pTZ18-citE and pNM-cit.

To verify that the sequence of the fragment of 350 nucleotides actaully corresponded to that of the cit gene, the nucleotide sequence of this fragment was determined starting from the recombinant plasmid pTZ 18-citE. The sequence reported in FIG. 6 [SEQ ID NO. 7] is identical to that already published (Nordling et al., 1990; Arai et al., 1990) with the exception of a substitution from T to C in position 102 of the coding sequence.

The sequence of this zone of the gene of Ps. aeruginosa was then verified before amplification, and the substitution resulted to be present, excluding thereby an error of polymerase during the amplification process.

This mutation, which conversely has no influence on correct translation, can be explained on the basis of the naturally frequent onset of silent mutations, considering that the Ps. aeruginosa strain we used for isolation of the nir and cit genes differed from that used by Nordling et al. (1990) and Arai et al. (1990).

### Methods Employed

Except for the cases explicitly indicated with specific references, all the methods of molecular genetics employed herein are described in Sambrook et al. (1989).

1) Isolation of the cit gene by PCR.

In order to isolate the citT gene, the oligonucleotides reported in FIG. 2 were designed. The sequences of the oligonucleotides are reported in this figure [SEQ ID Nos. 5 and 6] and the function of each subsequence is specified (i.e. cleaving site for restriction enzyme, linker, ribosome binding site or RBS; coding sequence of the cytochrome).

The two oligonucleotides are complementary to the 5′-terminal end and to the 3′-terminal end of the cit gene, respectively and were used for the cloning in vector pNM185 and also for the cloning in the sequence vector pTZ18. The PCR reaction was performed using 1 and 10 ng of the recombinant plasmid pEMBL18-NR in the presence of 50 pmoles of each of the two specific primers; the Taq polymerase used is Amplitaq (Perkin Elmer Cetus Corp.).

The reaction conditions were as follows:

a) 5′ at 95° C.

b) 1′ at 94° C.

c) 1′ at 58° C.

d) 2′ at 72° C.

The b-d steps were repeated for 30 consecutive cycles and the mixture was subsequently equilibrated at 35° C. for 15′.

Then, one tenth of the reaction was analysed on a 1.2% agarose gel in TBE.

The result, shown in FIG. 3, reveals the presence of an amplified fragment of approximately 350 nucleotides; this fragment was extracted from the gel and purified using the low melting point agarose procedure.

2) Cloning in the expression and sequence vectors.

The fragment of 350 nucleotides containing the cit gene was cloned in the following vectors, using the restriction sites described:

pNM185 in EcoRI site;

pTZ18 in EcoRI site.

For this purpose the vectors were digested with the above enzyme (Biolabs) according to the instructions supplied by the manufacturer, digestion was controlled on 1% agarose gel in TBE; in parallel, the same digestions with EcoRI were carried out on the fragment deriving from PCR. Both the DNAs (vectors and inserts) were purified by means of the low melting point agarose procedure. The recombinant plasmids were obtained by means of ligation in the presence of T4 DNA Ligase (Biolabs) for 12 hours at 16° C.; the constructs were then inserted by transformation of the E. coli JM 109 cells made competent by a treatment with CaCl₂.

For the isolation of the recombinant plasmid pNM-cit approximately 200 colonies deriving from the transformation were replica-plated onto LB plates containing Kanamycin (30 g/ml) and onto nylon filters (Hybond N, Amersham). The colonies were grown at 37° C. for 12 hours. The filters were treated with a denaturing solution to denature the plasmid DNA and hybridized with cit gene isolated and labelled by the random priming technique with P³²-ATP. The hybridization conditions used are those recommended by the manufacturer Amersham for Hybond N filters.

The positive colonies (see FIG. 7) were isolated from the replicated plate and from these colonies the plasmid DNA was prepared; the presence of the fragment of 350 bp was further checked by EcoRi digestion.

For isolation of the recombinant plasmid pTZ18-citE the plasmid DNA was directly prepared starting from the colonies deriving from transformation: the presence of the insert was checked by EcoRI of digestion (see FIG. 8).

3) Determination of the complete gene cit sequence.

The nucleotide sequence of the cit gene was determined by the Sanger method using the reagents Sequenase version 2.0 (USB); to eliminate the problems of compression deriving from the high content in GC of DNA of Ps. aeriginosa, the sequence reactions were carried out both with dGTP and 7AZA-dGTP. The sequence was resolved by means of electrophoresis on 6% urea/polyacrylamide gel.

### EXAMPLE 3. Expression of the cit gene in Ps. putida.

The presence of the cit gene in the previously isolated operon (Silvestrini et al., 1989) laid the basis for carrying out the expression of the cytochrome in a bacterial species, Ps. putida, similar to that of the origin (Ps. aeruginosa) and which was already used for the expression of the nir gene present in the same operon (Silvestrini et al., 1992). This bacterial species was chosen since, despite being correlated to the native species, it normally grows in aerobic conditions and presents limited nutritional requirements, making it possible its growth in the fermenter with limited costs. In addition, the expression of high levels of type c cytochrome in E. coli was so far carried only for a different cytochrome c (Ubbink et al., 1992), using conditions of growth in partial anaerobiosis which limit the possibility of expansion on a large scale of the growth itself.

The expression vector containing the cit gene (plasmid pNMcit), was introduced by transformation into strain of Ps. putida PaW340: enrichment in type c cytochromes was tested by differential spectra between the oxidized form and the reduced form of the iron atoms present in the hemoproteins on total cell lysed.

To verify whether the protein produced effectively corresponded to cytochrome C₅₅₁, a protein of apparent molecular weight of 9-kDa in SDS-PAGE gel and with electrophoretic mobility in denaturing conditions identical to that of the native cytochrome from Ps. aeruginosa, was purified to homogeneity (Parr et al. 1976).

This protein was further controlled by determination of the N-terminal sequence (first 35 residues): this sequence perfectly corresponded to the sequence of the mature cytochrome C₅₅₁ previously published (Ambler, 1963) and shows that the protein, coded at the gene level as a pre-protein with a signal sequence of 22 aminoacids needed for the translocation in the in bacterial periplasma (Nordling et al., 1990), is correctly processed also in the heterologous system of Ps. putida.

### Methods employed

Except for the cases explicitly indicated with specific references, all the methods of molecular genetics employed are described in Sambrook et al. (1989).

1) Transformation of PaW340 with the vector pNMcit.

The above described plasmid pNMcit was purified from the clone of E.coli JM109, in which it was previously inserted, using the method of alkaline lysis. The DNA thus obtained (10-100 ng) was introduced by transformation into PaW340 cells made competent by the CaCl₂ and MgCl₂ method (Lederberg and Cohen, 1974); the transformants were selected at 30° C. on LB medium containing 30 g/ml of kanamycin. The presence of the plasmid was controlled in the transformants extracting the DNA by the method of alkaline lysis: the clone containing the recombinant plasmid was called PaW340-pNMcit.

2) Induction of the cytochrome expression.

Plasmid pNMcit contains the cit gene cloned under control of the promoter Pm: the expression can be induced by stimulating the transcription from this promoter with the inductor m-toluate (Mermod et al., 1986).

Induction was carried out as is described below.

With a colony isolated from clone PaW340-pNMcit on solid LB medium +30 g/ml of kanamycin, a 5 ml pre-culture was inoculated into the same liquid medium and allowed to grow at 30° C. for approximately 6-8 hours.

The pre-culture was then diluted (1:100) in 100 ml of LB liquid medium +30 g/ml of kanamycin (non-induced control) and, in parallel, on the same medium containing 0.5 nM m-toluate (induced sample); the culture was grown for 16-18 hours at 30° C. The same induction assay was carried out for strain PaW340 containing only the plasmid pNM185 (without the cit gene) as control.

The cells were collected by centrifugation at 12,000 rpm for 20′at 4° C.

3) Differential spectra

The cells deriving from the above described induction experiment were resuspended in 0.1 M phosphate buffer pH 7.0 (4 ml/g cells) and sonicated in ice 5×1′.

To the supernatant of the sonication recovered by centrifugation (20′ at 12,000 rpm) potassium ferric cyanide was added to oxidize the iron of hemoproteins possibly present; this oxidized extract was divided into equal volumes in spectrophotometric cells (1 cm optical path) and a baseline between 400 and 500 nm was recorded. In one of the samples sodium dithionite (solid) was subsequently added and the spectrum at the same wavelength was recorded.

4) Purification of the cytochrome

The cells of the strain PaW340-pNMcit are grown on a large scale according to the methods described above for the induced samples: typically, from 1.5 liter cultures ( 6×250 ml in 2-liters beakers) approximately 10 g of wet cells are obtained.

The purification protocol is identical to that reported by Parr et al. (1976) up to gel filtration chromatography through a Sephadex G-75 column. At this point the fractions with slow chromatographic mobility (in which the presence of type c cytochromes are checked spectrophotometrically) are collected and pooled; these fractions are brought to pH 3.9 with the addition of acetic acid and centrifuged at 12,000 rpm for 20′ to eliminate any precipitates. The supernatant is recovered and loaded onto a CM52 ion exchange column (Whatman) equilibrated with 50 mM ammonium acetate, pH 3.9; the column is subsequently washed with the same buffer and the protein is eluted with 50 mM ammonium acetate, pH 4.45.

The eluted fractions are analysed spectrophotometrically between 250 and 650 nm to determine the purity of the sample obtained; generally, for the native cytochrome this index of purity is determined from the ratio between Abs (550-570 nm) of the reduced form and the Abs (280 nm) of the oxidized form: this ratio must have a value of 1.14 for a 100% pure protein (Parr et al., 1976).

5) Determination of the N-terminal sequence

The analysis of the sequence was carried out using an Applied Biosystems model 470A sequencer in gas phase supplied by an Applied Biosystems model 120A PTH analyser for the determination of phenylthiohydantoin derivatives of aminoacids.

The sample is applied to glass fibre filters treated with trifluoroacetic acid, coated with polyprene and pre-washed according to the instructions given by the manufacturing company.

### EXAMPLE 4. Production of cytochrome C₅₅₁ from Ps. putida PaW340

This example describes the procedure set to produce the cytochrome C₅₅₁ starting from the expression system pNMcit in PaW340 applying an optimized fermentation protocol. The subsequent purification procedure allows to obtain preparations of cytochrome C₅₅₁ with a high degree of purity in only two steps. The fermentation protocols of the strain PaW340-pNMcit and subsequent purification were optimized in order to be applicable on large scale and obtain a higher production yield. In order to obtain significant amounts of biomass and high levels of specific expression the fermentation procedure was optimized in preliminary experiments in which the following parameters were considered: composition of the culture medium, temperature, glucose concentration and partial pressure of oxygen. The optimized conditions were applied later on to 10-liter fermenters and the cellular biomass obtained was used for the extraction of cytochrome C₅₅₁. For purification, a new procedure was developed, characterized by a limited number of steps and an elevated overall yield, easily applicable on industrial scale. The recombinant protein obtained according to the new purification procedure resulted to be pure from the physicochemical standpoint and functionally active.

### Methods employed

1) Optimization of the fermentation conditions

The best conditions for the growth of strain PaW340-pNMcit were studied by evaluating the effect of the growth conditions on the levels of biomass and on the expression of the recombinant protein so as to ensure the achievement of significant levels of volumetric productivity and specific expression (mg of cytochrome C₅₅₁/gram of cells).

As reported in Table 1, the best results are those obtained in the conditions of test 6, in which approximately 20 grams of wet biomass/liter of fermentation were recovered with levels of cytochrome C₅₅₁ of approximately 1 milligram/gram of cells.

TABLE 1
Fermentation of the recombinant strain PaW340-pNMcit.

| | Operating conditions | | | | Results | | |
|---|---|---|---|---|---|---|---|
| | | | | % | Bio- | | cyt C₅₅₁ |
| Test | | Temp. | Glucose | Oxygen | mass | cyt C₅₅₁ | mg/g |
| No. | Medium | ° C. | % | satur. | g/l | mg/l | biomass |
| 1 | SAEM | 30 | — | 40 | 118 | 11.8 | 0.10 |
| 2 | TBM | 30 | — | 0 | 6 | 4.2 | 0.70 |
| 3 | TBM | 30 | 0.2 | 5 | 31 | 4.4 | 0.14 |
| 4 | TBM | 30 | 0.2 | 0 | 7 | 0.8 | 0.11 |
| 5 | F120 | 30 | 0.2 | 2 | 41 | 17.3 | 0.42 |
| 6 | F120 | 32 | 0.4 | 2 | 21 | 19.3 | 0.92 |
| 7 | F120 | 32 | 0.4 | 2 | 19 | 19.5 | 1.03 |
| 8 | F120 | 32 | 0.4 | 2 | 18 | 17.0 | 0.94 |

The optimized conditions selected for the production are characterized mainly by the following parameters:

culture medium F120 at pH 7

temperature of 32° C.

O₂ level maintained at 2% of saturation

The reproducibility of the optimized fermentation protocol, which is a condition essential for its application on a preparative scale, was confirmed by replications carried out in 10-liter fermenters (tests 7 and 8 reported in table 1) and on a higher scale.

2. Fermentation

a) Composition of the culture medium

LK medium (Luria-Bertani with kanamycin)

| | | |
|---|---|---|
| Bactotryptone | 20 | g/l |
| Yeast extract | 10 | g/l |
| NaCL | 10 | g/l |
| Kanamycin | 50 | mg/l |
| Medium F120: | | |
| Hydrolyzed casein | 18 | g/l |
| Yeast extract | 36 | g/l |
| KH₂PO₄ | 0.58 | g/l |
| K₂HPO₄ | 3.14 | g/l |
| Glucose | 4 | g/I |
| Toluic acid | 0.68 | g/l |
| Kanamycin | 50 | mg/l |

b) Vegetative phase

To two 1-liter beakers containing 250 ml of LK medium, 0.5 ml of a suspension of the recombinant strain PaW340-pNMcit was added and the cultivation was carried out at 30° C. on an oscillating shaker at 160 rpm for 16 hours. The bacterial culture obtained was used as inoculum for the productive phase.

c) Productive phase

10-liter fermenters were used containing 5 liters of F 120 medium at pH 7 inoculated with the product of the vegetative phase. Fermentation was carried out at 32 1° C., with stirring adjusted to 300 rpm and an airflow of 0.25 liters/liter of culture. During fermentation stirring was automatically modified so as to maintain the level of oxygen at 2% saturation. The productive phase of fermentation lasted for approximately 9 hours, until a final cellular growth corresponding to an optical density measured at 600 nm of approximately 20 units was obtained. At the end of fermentation the cells collected by centrifugation typically corresponded to approximately 100 grams of wet weight.

3. Purification

The purification operations were carried out at 4° C.

The biomass obtained from a 5-liter fermentation (approx. 100 grams of wet weight) was resuspended in 1200 ml of 0.1 M tris-1 mM phenyl-methylsulfonylfluoride-I mM EDTA buffer, pH 7 and the cells were disintegrated by two passages in a mechanical homogenizer (Type APV-Rainin) at the pressure of approximately 800 bar. Alternatively, cell rupture could be obtained by sonication. The cellular homogenate was centrifuged at 7000 g for 30 minutes, the supernatant was recovered, brought to pH 4.0 by the addition of diluted acetic acid and centrifuged again. To the pink coloured supernatant 2 ml of 5% K₂Fe (CN)₆ was added to oxidize cytochrome C₅₅₁, and the solution was dialysed for approximately 4 hours against a 20 mM acetate buffer solution pH 4 (buffer A). The dialysed solution was loaded onto a chromatographic column containing a 200 ml CM-Sepharose Fast Flow resin bed (Pharmacia Biotech company) and pre-equilibrated with buffer A. The column was washed with 1 liter of buffer A and subsequently eluted at the following conditions: linear gradient from 100% buffer A to 35% of buffer B (20 mM sodium acetate-0.5 M NaCI - pH 4.0) in 60 minutes followed by an isocratic elution for 30 minutes with a phase consisting of 65% buffer A and 35% buffer B and a linear gradient from 40% to 100% buffer B in 30 minutes The fractions containing cytochrome C₅₅₁ characterized by a slight pink color were controlled by spectrophotometric analysis and reversed-phase high pressure liquid chromatography (RP-HPLC) and pooled on the basis of the results of the analysis. The preparations of cytochrome C₅₅₁ were stored at +4° C. or, alternatively, lyophilized after dialysis against 10 mM ammonium acetate buffer, to obtain a preparation of the recombinant protein in the solid form.

A typical example of the results obtained in the purification procedure is reported in the following scheme:

| | Cytochrome C₅₅₁ | Yield |
|---|---|---|
| Purification stage | mg | % |
| Cellular homogenate | 90 | 100 |
| Supernatant after acid | 76.5 | 85 |
| precipitation | | |
| Pool after CM-Sepharose | 58.5 | 65 |
| colony | | |

The preparations of cytochrome C₅₅₁ obtained according to the purification protocol described presented typically a purity greater than 90% when they were examined by reversed-phase high pressure liquid chromatography (FIG. 9), isoelectric focusing (FIG. 10) and polyacrylamide gel electrophoresis (FIG. 1).

### EXAMPLE 5.

Characterization of recombinant cytochrome C₅₅₁ in the native form Recombinant Cytochrome C₅₅₁ obtained by the purification described in Example 4 was characterized to establish whether the protein obtained was effectively identical to the native form purified from Ps. aeruginosa.

Some of the main characteristics of this hemoprotein are common to other type c cytochromes: firstly, the prosthetic group, a porphyrin containing an atom of iron, that is covalently bound to the protein component by means of two thioether bonds with two cysteine residues of the protein (C12 and C15, Ambler, 1963). The presence of these specific covalent bonds between heme and protein, missing in the other types of cytochromes (a, b, d, etc.) is in itself symptomatic of the correct conformation assumed by the protein.

In addition, the experimental measure of the mass obtainable with high precision by means of mass spectrometry techniques, constitutes a direct confirmation of the molecular structure of the recombinant cytochrome C₅₅₁ with regards to the integrity of the polypeptide chain as well as formation of the covalent bond with heme.

A correct tridimensional structure influences also the spectroscopic characteristics of the chromophor (heme); it, in fact, depends not only on the intrinsic properties of light absorption of this part of the heme but also on the interaction of this chromophor with the surrounding environment, i.e. the protein component.

Also the information obtained in circular dichroism experiments in the visible zone can be correlated with the integrity of the molecule around the chromophor, which also in this case is the heme.

Thus the characterization of the recombinant cytochrome was carried out by electrophoresis on gel as well as through extensive spectroscopic characterization, optical and circular dichroism. The following were analysed:

the presence of the covalent bond between the protein and heme, by means of electrophoresis in denaturing conditions and heme-specific staining;

the spectra of the recombinant cytochrome in both the oxidized and reduced form (FIG. 12);

the presence of a typical spectroscopic pH-dependent variation, already largely characterized in native cytochrome C₅₅₁ purified from Ps. aeruginosa (Silvestrini et al., 1981) (FIG. 13);

the spectra of circular dichroism in the visible zone, by near and deep UV for the recombinant cytochrome in the oxidized form (FIGS. 14 and 16);

the spectra of circular dichroism in the visible zone, by near UV for the recombinant cytochrome in the reduced form (FIG. 15).

This characterization has made it possible to establish that cytochrome C₅₅₁ produced by the recombinant DNA in Ps. putida is in the native form and that, as regards the characteristics of the molecule analysed by us, it is indistinguishable from the native cytochrome C₅₅₁ purified from Ps. aeruginosa.

### Methods Employed

1) Electrophoresis

The recombinant cytochrome was analysed by electrophoresis in denaturing conditions according to the method of electrophoresis in Tricine-SDS (Schagger and Von Jagow, 1987). The gel was stained with Coomassie Blue and with a heme-specific stain according to the benzidine method (Thomas et al., 1976).

The result of this experiment indicates that the recombinant cytochrome possesses a heme group covalently bound, since it does not dissociate from the protein in denaturing conditions.

2) Optical Spectroscopy

Spectroscopic analysis was performed using a Cary 219 double beam spectrophotometer (Varian). The sample of recombinant cytochrome, in 0.1 M Na/phosphate buffer, pH 7.0 was analysed between 260 and 600 nm (in the oxidized form) and between 380 and 600 nm (in the reduced form). The protein deriving by purification is found in the oxidized form; the reduced form is obtained by the addition of solid sodium dithionite.

FIG. 10 shows the spectra of the oxidized and reduced form of recombinant hemoprotein: both the spectra show the characteristic peaks of absorption at 280, 410 and 530 nm (oxidized C₅₅₁) and at 417, 520 and 551 (reduced C₅₅₁) already described for the native C₅₅₁ purified from Ps. aeruginosa (Horio et al., 1960).

The presence of pH-dependent spectroscopic variation of the absorption peak at 551 nm , characteristic of native C₅₅₁ purified from Ps. aeruginosa (Silvestrini et al., 1981), was also verified: in this case the sample of recombinant cytochrome, initially at pH 5.5, was brought to pH 7.2 and to pH 10.0 by addition of 4 M NaOH. To each pH value the spectrum between 500 and 600 nm was recorded: FIG. 13 indicates that at alkaline pH, the absorption peak at 551 nm shifts towards longer wavelengths, with a simultaneous decrease of the maximum value of absorption. This characteristic spectroscopic variation (alkaline shift), absent in other type c cytochromes, is most likely correlated with the variation of the ionization state of a residue of the native cytochrome C₅₅₁ in proximity to the prosthetic group, which is recorded by heme itself.

3) Spectroscopy of Circular Dichroism

The experiments of circular dichroism were performed on a Jasco J500 spectropolarimeter complete with data analyser (Jasco model DP S00 N). In these experiments a solution of the recombinant cytochrome in 0.1 M Na/phosphate buffer, pH 7.0 was analysed between 200 and 600 nm (oxidized cytochrome) and between 380 and 600 nm (reduced cytochrome). Also in this case the reduction of heminic iron was obtained by the addition of solid sodium dithionite. To improve the quality of the signal, the spectra in the 200-450 nm zone were accumulated 4 times and those in the 450-600 nm zone twice. The result of these experiments, shown in FIGS. 14-16, indicate that the recombinant cytochrome is identical to native C₅₅₁, purified from Ps. aeruginosa; also in this case the recombinant protein has the characteristic of native C₅₅₁.

4) Determination of the Molecular Mass by Mass Spectrometry

The preparation of cytochrome C₅₅₁, dissolved in methanol-water-acetic acid (50:50:0.1) at the concentration of 0.2 mg/ml was injected at a flow rate of 2 microliters/minute into a single quadrupole Hewlett-Packard model 5989A mass spectrometer connected to a Hewlett-Packard model 59987A electro-spray interface.

The experimental value of the molecular mass (FIG. 17) gave a value of 9309.97 Da corresponding, within an interval of variation of 1 Da, to the value calculated summing the molecular weights of the apoprotein and heme covalently bound with two thioether bonds and containing an iron atom.

### Bibliographic References

Ambler R. P. (1963) Biochem. J. 46. 1289-1301.

Arai H., Sanbongi Y., Igarashi Y. and Kodama T. (1990), FEBS Letters 261. 196-198.

Horio T., Higashi T., Sasagawa M., Kusai K., Nakai M. and Okunuki K. (1960), Biochem. J., 77, 194-201.

Lederberg E. M. and Cohen S. N. (1974), J. Bacteriol., 119, 1072-1074.

Mead D. A. et al. (1986), Prot. Enin. 1, 67.

Mermod N., Ramos J. L., Lehrbach P. R. and Tirrunis K. (1986), J. Bacteriol, 167, 447-454.

Mullis and Faloona (1987), Meth. Enzymol. 155, p. 335.

Nordling M., Young. S., Karlsson B. G. and Lundberg L. G. (1990), Febs Letters 259, 230-232.

Parr S. R., Barber D., Greenwood C., Phillips B. W. and Melling J. (1976), Biochem. J., 157, 423-430.

Sambrook J., Fritsch E. F. and Maniatis T. (1989), “Molecular Cloning: a Laboratory manual”, second edition, Cold Spring Harbor Laboratory Press.

Sanger F., Nicklen S. and Coulson A. R. (1977), Proc. Natl. Aca. Sci. USA, 78, 5463-5467.

Schagger H. and von Jagow G. (1987), Anal. Biochem., 166, 368-379.

Silvestrini M. C., Galeotti Barra D., Gervais M., Schinina E., Barra D., Bossa F. and Brunori M. (1989), Febs Letters, 254, 33-38.

Silvestrini M. C., Cutnrzzolä F., D'Alessandro R., Brunori M., Fochesato N. and Zennaro E. (1992), Biochem. J., 285, 661-666.

Silvestrini M. C., Brunori M., Wilson M.T. and Darley-Usmar V. (1981), J. Inorg. Biochem., 14, 327-338.

Thomas P. E., Ryan D. and Levin W. (1976), Analyt. Biochem., 75, 168-176.

Ubbink M., Van Beeumen J. and Canters G. W. (1992), J. Bacteriol. 174, 3707-3714

1. Recombinant process for the production of a hemoprotein with the ability of transporting electrons comprising the sequence of cytochrome C₅₅₁ of Pseudomonas aeruginosa, characterized in that this hemoprotein is produced in Pseudomonas putida.

2. Procedure according to claim 1 in which the hemoprotein possesses a signal sequence able to translocate the cytochrome C₅₅₁ in the cellular compartment where it exerts its functional activity.

3. DNA molecule coding for a hemoprotein with the ability of transporting electrons comprising the following sequence

| | |
|---|---|
| GAA GAC CCC GAA GTG CTG TTC AAG AAC AAG GGC TGC GTG GCC | [SEQ ID NO.1]: |
| TGC CAT GCC ATC GAC ACC AAG ATG GTC GGC CCG GCC TAC AAG GAC | |
| GTC GCC GCC AAG TTC GCC GGC CAG GCC GGC GCG GAA GCG GAA | |
| CTC GCG CAG CGG ATC AAG AAC GGC AGC CAG GGC GTC TGG GGC | |
| CCG ATC CCG ATG CCG CCG AAC GCG GTC AGC GAC GAC GAG GCG | |
| CAG ACC CTG GCG AAG TGG GTC CTG TCG CAG AAA TGA | |

4. DNA molecule comprising the sequence of claim 3 immediately preceded by the following signal sequence

| | |
|---|---|
| ATG AAA CCG TAC GCA CTG CTT TCG CTG CTC GCC ACC GGC ACC CTG | [SEQ ID NO. 2]: |
| CTC GCC CAG GGC GCC TGG GCC | |

5. DNA molecule as reported in FIG. 6 of the attached drawings.

6. Plasmid expression vector comprising a DNA molecule according to any of the claims from 3 to 5.

7. Bacterial host transformed by a plasmid expression vector according to claim 6.

8. Host according to claim 7 consisting of a strain of Pseudomonas putida.


SEQUENCE LISTING (7 sequences)

SEQ ID NO: 1 - DNA, 249 bp, Pseudomonas aeruginosa
gaagaccccgaagtgctgttcaagaacaagggctgcgtggcctgccatgccatcgacaccaagatggtcggcccggcctacaaggacgtcgccgccaagttcgccggccaggccggcgcggaagcggaactcgcgcagcggatcaagaacggcagccagggcgtctggggcccgatcccgatgccgccgaacgcggtcagcgacgacgaggcgcagaccctggcgaagtgggtcctgtcgcagaaatga

SEQ ID NO: 2 - DNA, 66 bp, Artificial Sequence
  Description of Artificial Sequence DNA leader sequence
atgaaaccgtacgcactgctttcgctgctcgccaccggcaccctgctcgcccagggcgcctgggcc

SEQ ID NO: 3 - DNA, 19 bp, Artificial Sequence
  Description of Artificial Sequence synthetic oligonucleotide 28
acgacaagaccctgaagct

SEQ ID NO: 4 - DNA, 20 bp, Artificial Sequence
  Description of Artificial Sequence synthetic oligonucleotide 29
tacgcactgctttcgctgct

SEQ ID NO: 5 - DNA, 42 bp, Artificial Sequence
  Description of Artificial Sequence synthetic oligonucleotide NM-1
cggcgcgaattcgaggaaccgtgatgaaaccgtacgcactgc

SEQ ID NO: 6 - DNA, 40 bp, Artificial Sequence
  Description of Artificial Sequence synthetic oligonucleotide NM-2
cggcgcgaattctcatttctgcgacaggacccacttcgcc

SEQ ID NO: 7 - DNA, 338 bp, Artificial Sequence
  Description of Artificial Sequence DNA (genomic)
gaattcgaggaaccgtgatgaaaccgtacgcactgctttcgctgctcgccaccggcaccctgctcgcccagggcgcctgggccgaagaccccgaagtgctgttcaagaacaagggctgcgtggcctgccatgccatcgacaccaagatggtcggcccggcctacaaggacgtcgccgccaagttcgccggccaggccggcgcggaagcggaactcgcgcagcggatcaagaacggcagccagggcgtctggggcccgatcccgatgccgccgaacgcggtcagcgacgacgaggcgcagaccctggcgaagtgggtcctgtcgcagaaatgagaattc